(19) United States
(12) Patent Application Publication
(10) Pub. No.: US 2008/0188931 A1
(43) Pub. Date: Aug. 7, 2008

(54) Cricoid wedge implant

(75) Inventor: Tack-Kyun Kwon, Seoul (KR)

Correspondence Address: RABIN & Berdo, PC, 1101 14TH STREET, NW, SUITE 500, WASHINGTON, DC, 20005, US

(73) Assignee: SEOUL NATIONAL UNIVERSITY HOSPITAL, Jongno-Gu (KR)

(21) Appl. No.: 11/639,144

(22) Filed: Dec. 15, 2006

(30) Foreign Application Priority Data
Nov. 1, 2006 (KR) 10-2006-0107434

(51) Int. Cl. A61F 2/20

(52) U.S. Cl. 623/9

(57) ABSTRACT

The present invention relates to an implant inserted into a vocal fold and, more particularly, to a cricoid wedge implant that medializes an arytenoid cartilage of a patient endoscopically to treat vocal fold paralysis.

The cricoid wedge implant of the present invention comprises a disc type head portion established on one end thereof, a needle portion, of which a section is inclined, provided on the other end thereof; and a tube type body in which a hole is penetrated from the head portion to the needle portion.

The present invention medializes the arytenoid cartilage endoscopically without an invasive operation and thereby has advantages in that it takes a shorter time required for the treatment than the conventional method, in which an invasive arytenoid addition is operated to the patient who cannot be cured only with an injection laryngoplasty, and it will not cause any complications.

### CROSS-REFERENCE TO RELATED APPLICATION

This application claims the benefit of Korean Patent Application No. 10-2006-0107434, filed on Nov. 1, 2006, the entire disclosure of which is hereby incorporated by reference.

### BACKGROUND OF THE INVENTION

1. Field of the Invention

The present invention relates to an implant inserted into a vocal fold and, more particularly, to a cricoid wedge implant that medializes an arytenoid cartilage of a patient endoscopically to treat vocal fold paralysis.

2. Description of Related Art

Vocal folds, an important structure of a larynx that is a vocal apparatus of the human body, are positioned on both sides of the larynx. The vocal folds are abducted when breathing and adducted when speaking.

Especially, the vocal folds are an essential structure to speak and, if the vocal folds are abnormal, a coarse voice occurs to cause difficulties in communicating with people and the abnormal vocal folds are more closely connected to swallowing.

Vocal fold paralysis means a state where the vocal folds' movement is paralyzed due to abnormal nerves that make the vocal folds to move and causes various diseases associated with the same.

Unilateral vocal fold paralysis is a result of dysfunction of the vocal fold on one side and has the highest frequency. In this case, even if the movement of the opposite vocal fold is normal, the vocal folds on both sides do not stick together and thereby a vocal fold gap is formed. Accordingly, it is impossible to make the vibration of the vocal folds and speak with a normal voice.

Moreover, since a function of preventing saliva or food from getting into the airway is deteriorated among various functions of the vocal folds, a dysphagia may be induced due to frequent aspirations and, if severe, a fatal complication such as aspiration pneumonia may be caused.

Typical therapies for unilateral vocal fold paralysis include a thyroplasty and an arytenoid adduction. Since these two methods have mechanisms different from each other, the best result can be obtained if the two surgical operations are made at the same time.

Recently, as non-invasive operation methods have attracted attention, an injection laryngoplasty corresponding to the thyroplasty has been widely used to be a great help to patients. Such an operation uses a method in which a foreign body is injected into the vocal fold using a needle in regional anesthesia and has advantages in that it does not injure the skin and in terms of the treatment time and the incidence of complications compared with the thyroplasty.

However, since a non-invasive operation corresponding to the arytenoid adduction has not yet been developed, if the unilateral vocal fold paralysis is not completely treated by the injection laryngoplasty, an invasive arytenoid adduction operation should be performed additionally.

### SUMMARY OF THE INVENTION

The present invention has been contrived taking the above-described circumstances into consideration, and an object of the present invention is to provide a cricoid wedge implant that medializes an arytenoid cartilage endoscopically.

To accomplish the above object of the present invention, there is provided a cricoid wedge implant comprises a disc type head portion established on one end thereof, a needle portion, of which a section is inclined, provided on the other end thereof, and a tube type body in which a hole is penetrated from the head portion to the needle portion.

Here, a side hole may be established on the side surface of the body to fix the cricoid wedge implant.

Moreover, a screw thread is established on the outer circumference of the body to fix the cricoid wedge implant.

Furthermore, the cricoid wedge implant of the present invention is made of titanium that is friendly to the human body.

Other objects and advantages of the present invention will be described hereinafter and become more apparent from preferred embodiments of the present invention. Moreover, the objects and advantages of the present invention can be embodied by means and combinations set forth in the appended claims.

### BRIEF DESCRIPTION OF THE DRAWINGS

FIG. 1 is a cross-sectional view depicting a cricoid wedge implant in accordance with a preferred embodiment of the present invention; and

FIG. 2 is a cross-sectional view depicting a cricoid wedge implant in accordance with another preferred embodiment of the present invention.

### DETAILED DESCRIPTION OF THE INVENTION

Hereinafter, preferred embodiments of the present invention will now be described in detail with reference to the attached drawings.

Hereinafter, preferred embodiments of the present invention will now be described with reference to the accompanying drawings. First of all, terms and words used in the specification and the claims should be interpreted not in a limited normal or dictionary meaning, but to include meanings and concepts conforming with technical aspects of the present invention, based on the fact that the inventor may appropriately define a concept of a term to describe his/her own invention in a best way.

Therefore, the configurations described in the specification and drawn in the figures are just most preferred embodiments of the present invention, not to show all of the technical aspects of the present invention. So, it should be understood that there might be various equalities and modifications to be replaced with them at this point of time when the present application is filed.

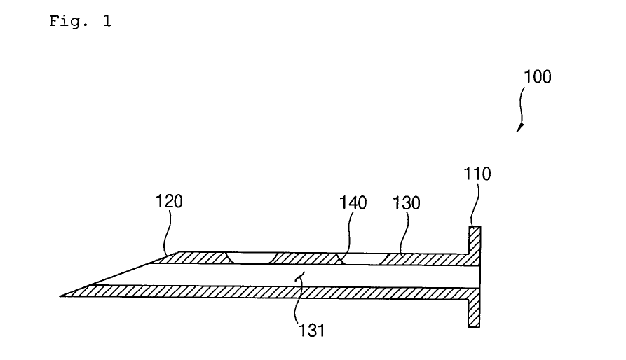

In FIG. 1, a cross-sectional view of a cricoid wedge implant in accordance with a preferred embodiment of the present invention is depicted. As depicted in FIG. 1, a disc type head portion 110 for preventing the cricoid wedge implant 100 in accordance with the preferred embodiment of the present invention from being inserted over a predetermined depth is established on an end thereof.

Moreover, a needle portion 120 for facilitating the implantation is formed on the other end of the cricoid wedge implant 100 and a hole 131 penetrating from the head portion 110 to the needle portion 120 is established inside a tube type body 130, thus constituting the cricoid wedge implant 100.

Furthermore, a side hole 140 is provided on the side surface of the body 130 of the cricoid wedge implant 100 in accordance with the preferred embodiment of the present invention.

After implanting the cricoid wedge implant 100 of the present invention into the vocal fold of a patient, the patient's cartilage tissue grows along with the side hole 140 to fix the cricoid wedge implant 100.

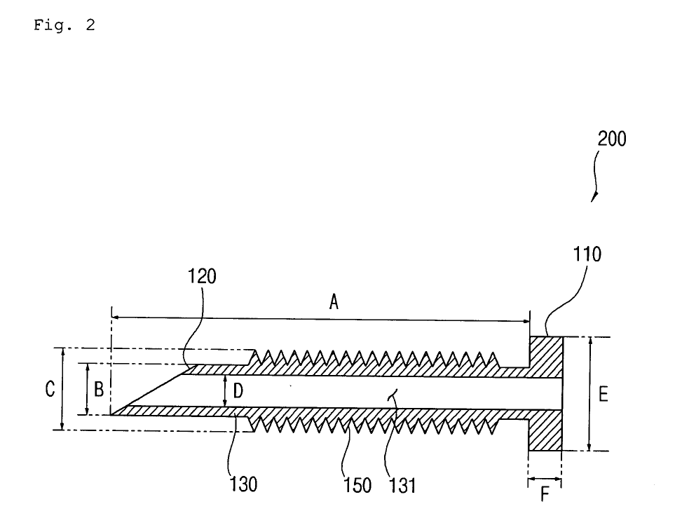

Meanwhile, as depicted in FIG. 2, a cricoid wedge implant 200 in accordance with another embodiment of the present invention may includes a screw thread 150 established on the outer circumference of the body 130 based on the ossification degree of the cricoid cartilage.

In general, the cricoid wedge implant desirably has a length A of 12mm excluding the head portion 110, an outer diameter B of the body 130 of 1.2-1.4 mm (an outer diameter C of 1.4-1.6 mm if including the screw treads 150), an inner diameter D of 0.8-1.0 mm, an outer diameter E of the head portion 110 of 1.6-1.8 mm and a length F of the head portion 110 of 2-4 mm; however, not limited thereto, but those sizes may be changed as much as possible based on the size of cartilages of patients.

Moreover, the cricoid wedge implant of the present invention may be made of stainless; however, it is more desirable to make the cricoid wedge implant with titanium that is more friendly to the human body.

Hereinafter, the process of implanting the cricoid wedge implant of the present invention will be described. In the description, the same elements that perform the same functions are denoted by the same reference numerals in FIGS. 1 and 2.

First, the larynx of the patient is exposed through the mouth, the mucosa of cricoarytenoid articulation is incised and, then, the muscular process of cricoarytenoid cartilage is checked.

Subsequently, with pushing the muscular process, a guide needle penetrating through the hole 131 in the body 130 is inserted therein using a specific insertion tool.

Here, the guide needle is to prevent the cricoid wedge implant 100 or 200 of the present invention from being inserted in the corresponding cartilage in a direction that the operation surgeon does not want. That is, after inserting the guide needle into a position that the operation surgeon aims, i.e., into the object of the operation, the guide needle is inserted into the hole 131 of the cricoid wedge implant 100 or 200.

Here, it is preferable that the guide needle used for implanting the cricoid wedge implant 100 or 200 of the present invention be inserted therein using an insertion tool including a ratchet that is controlled so that the guide needle is not bent but inserted into the object of the operation little by little.

Subsequently, the cricoid wedge implant 100 or 200 is implanted into the object of the operation along with the guide needle inserted into the hole 131 of the cricoid wedge implant 100 or 200.

Last, the guide needle is removed to complete the operation, leaving the cricoid wedge implant 100 or 200.

As described above, the present invention medializes the arytenoid cartilage endoscopically without the invasive operation and thereby has advantages in that it takes a shorter time required for the treatment than the conventional method, in which the invasive arytenoid addition is operated to the patient who cannot be cured only with the injection laryngoplasty, and it will not cause any complications.

As described above, although the present invention has been described and illustrated with reference to preferred embodiments and drawings, it should be understood that various modifications and variations of the present invention can be made thereto by those skilled in the art without departing from the spirit and the technical scope of the present invention as defined by the appended claims.

What is claimed is:

1. A cricoid wedge implant comprises:
a disc type head portion established on one end thereof,
a needle portion, of which a section is inclined, provided on the other end thereof; and
a tube type body in which a hole is penetrated from the head portion to the needle portion.

2. The cricoid wedge implant as recited in claim 1,
wherein a side hole is established on the side surface of the body.

3. The cricoid wedge implant as recited in claim 1,
wherein a screw thread is established on the outer circumference of the body.

4. The cricoid wedge implant as recited in claim 2,
wherein the cricoid wedge implant is made of titanium.

5. The cricoid wedge implant as recited in claim 3,
wherein the cricoid wedge implant is made of titanium.
